(19) United States
(12) Patent Application Publication
(10) Pub. No.: US 2022/0310263 A1
(43) Pub. Date: Sep. 29, 2022

(54) TREATMENT PROTOCOL FOR ASSESSING AND MANAGING PAIN, BASED ON THE PATIENT'S RISK OF OPIOID MISUSE, ABUSE, AND/OR ADDICTION, AND METHOD OF USE

(71) Applicants: Larry B. Gelman, Sleepy Hollow, IL (US); Glenn B. Gelman, Crystal Lake, IL (US)

(72) Inventors: Larry B. Gelman, Sleepy Hollow, IL (US); Glenn B. Gelman, Crystal Lake, IL (US)

(21) Appl. No.: 17/656,466

(22) Filed: Mar. 25, 2022

Related U.S. Application Data
(60) Provisional application No. 63/200,733, filed on Mar. 25, 2021.

(51) Int. Cl. G16H 50/30; A61B 5/00; G16H 50/20; G16H 20/10

(52) U.S. Cl. CPC G16H 50/30; A61B 5/4842; G16H 50/20; G16H 20/10

(57) ABSTRACT

A pain management treatment protocol which assesses a patient's pain and related factors to susceptibility of opioid abuse, which evaluates a patient's capacity for pain treatments under multiple disciplines, and which further provides a rating for the administrating professional to apply the appropriate treatment.

### CROSS REFERENCE

This application is the non-provisional, utility application for, and claims the benefit of provisional application No. 63/200,733, titled “A Treatment Protocol for Assessing and Managing Pain Based on the Patient's Risk of Opioid Misuse, Abuse, and/or Addiction and Method of Use,” filed 25 Mar. 2021, by inventors, Larry B. Gelman, and Glenn B. Gelman, and is incorporated here by reference, including the specifications. This is not a conversion of the provisional, but a new application.

### BACKGROUND OF THE INVENTION

### Field of the Invention

The technology/technological field of the present invention is directed to prescribing the correct pain management treatment.

### Description of the Prior Art

The present invention is unlike other treatment protocols found in the technological field, which often are of limited focus to merely one discipline or approach on how to manage pain. For example, a medical physician is likely to limit their evaluation to the intensity and duration of the patient's pain, and biological factors. For example, a psychiatrist is likely to limit their evaluation to the mental health of the patient. For example, a statistician or sociologist studying opioid addiction is likely to limit their evaluation to socio-economic and demographic information about the patient. None of these examples will also weigh the insight of the administering professional as an additional point of data. There is a need for a protocol that assesses a patient's pain and related factors to susceptibility of opioid abuse, that which evaluates a patient's capacity for pain treatments under multiple disciplines, and further provides a rating for the administrating professional to apply the appropriate treatment.

### SUMMARY OF THE INVENTION

The present invention is a pain management treatment protocol which assesses a patient's pain and related factors to susceptibility of opioid abuse, which evaluates a patient's capacity for pain treatments under multiple disciplines, and which further provides a rating for the administrating professional to apply the appropriate treatment. The present invention is novel as it weighs the insight of the administering professional as an additional point of data. The present invention practically applies each of these different approaches through the protocol tool to select the safest and most effective pain management treatment with the least risk to the patient for opioid misuse, abuse, and/or addiction.

### DETAILED DESCRIPTION OF INVENTION

In this Specification, which includes the figures, claims, and this detailed description, reference is made to particular and possible features of the embodiments of the invention, including method steps. These particular and possible features are intended to include all possible combinations of such features, without exclusivity. For instance, where a feature is disclosed in a specific embodiment or claim, that feature can also be used, to the extent possible, in combination with and/or in the context of other aspects and embodiments of the invention, and in the invention generally. Additionally, the disclosed architecture is sufficiently configurable, such that it may be utilized in ways other than what is shown.

The purpose of the Abstract of this Specification is to enable the U.S. Patent and Trademark Office and the public generally, and especially the scientists, engineers and practitioners of the art who are not familiar with patent or legal terms or phrasing, to determine quickly from a cursory inspection the nature and essence of the technical disclosure of the application. The Abstract is not intended to be limiting as to the scope of the invention in any way.

In the following description, numerous specific details are given in order to provide a thorough understanding of the present embodiments. It will be apparent, however, to one having ordinary skill in the art, that the specific detail need not be employed to practice the present embodiments. On other instances, well-known materials or methods have not been described in detail in order to avoid obscuring the present embodiments. When limitations are intended in this Specification, they are made with expressly limiting or exhaustive language.

Reference throughout this Specification to “one embodiment”, “an embodiment”, “one example” or “an example” means that a particular feature, structure, or characteristic described in connection with the embodiment or example is included in at least one embodiment of the present embodiments. Thus, appearances of the phrases “in one embodiment”, “according to an embodiment”, “in an embodiment”, “one example”, “for example”, “an example”, or the like, in various places throughout this Specification are not necessarily all referring to the same embodiment or example. Furthermore, the particular features, structures, or characteristics may be combined in any suitable combinations and/or sub-combinations in one or more embodiments or examples.

The terms “comprises”, “comprising”, “includes”, “including”, “has”, “having”, “could”, “could have” or their grammatical equivalents, are used in this Specification to mean that other features, components, materials, steps, etc. are optionally present as a non-exclusive inclusion. For instance, a device “comprising” (or “which comprises”) components A, B, and C can contain only components A, B, and C, or can contain not only components A, B, and C but also one or more other components. For example, a method comprising two or more defined steps can be carried out in any order or simultaneously, unless the context excludes that possibility; and the method can include one or more other steps which are carried out before any of the defined steps, between two of the defined steps, or after all the defined steps, unless the context excludes that possibility.

Further, unless expressly stated to the contrary, “or” refers to an inclusive or and not to an exclusive or. For example, An embodiment could have optional features A, B, or C, so the embodiment could be satisfied with A in one instance, with B in another instance, and with C in a third instance, and probably with AB, AC, BC, or ABC if the context of features does not exclude that possibility.

Examples or illustrations given are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these example or illustrations are utilized will encompass other embodiments, which may or may not be given in this Specification, and all such embodiments are intended to be included within the scope of that term or terms. Language designating such nonlimiting examples and illustrations includes, but is not limited to “for example”, “for instance”, “etc.”, “or otherwise”, and “in one embodiment.”

The phrase “at least” followed by a number is used to denote the start of a range beginning with that number, which may or may not be a range having an upper limit, depending on the variable defined. For instance, “at least 1” means 1 or more.

In this specification. “a” and “an” and similar phrases are to be interpreted as “at least one” and “one or more.” In this specification, the term “may” or “can be” or “could be” is to be interpreted as “may, for example.” In other words, the term “may” is indicative that the phrase following the term “may” is an example of one of a multitude of suitable possibilities that may, or may not, be employed to one or more of the various embodiments.

The phrase “a plurality of” followed by a feature, component, or structure is used to mean more than one, specifically including a great many, relative to the context of the component. For example, “a plurality of reflectors” means more than one, and specifically includes more than a few and at least one embodiment of the invention includes hundreds of reflectors on one device.

It is the applicant's intent that only claims that include the express language “means for” or “step for” be interpreted under 35 U.S.C. § 112. Claims that do not expressly include the phrase “means for” or “step for” are not to be interpreted under 35 U.S.C. § 112.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purpose required by law, but otherwise reserves all copyright rights whatsoever.

Personal Information [Assessor to Rate Risk Factor Severity*]

The NICA PSYCHOBIOSOCIAL RISK ASSESSMENT: DIAGNOSTIC INTERVIEW and NICA OPIOID USE DISORDERS RISK ASSESSMENT: DSM-5-BASED CRITERIA (i.e., the NORA protocol) is designed to be completed, preferably, by a live (as opposed to an electronic) healthcare Assessor; not the Patient, in order for the Assessor to directly procure, face-to-face information of both a ‘quantitative’ and ‘qualitative’ nature from the Patient. Something as simple as requesting the Patient to provide their name is immediately fraught with potential difficulty. For example, what name is ‘officially’ considered by the Patient to be their legal name? Is there an alias being proffered? Has there been a divorce or other event or reason resulting in a name-change? And what about those Patients who may assert that their legal name is not identical to their idiosyncratically preferred name? If possible, it is advisable for the Assessor to obtain a legible photocopy of the Patient's identification (ID).

Emergency Contact [Assessor to Rate Risk Factor Severity*]

Similar challenges might conceivably be applicable to identifying a Patient's current address or emergency contact. Consequently, only by the Assessor's careful rendering to the Patient of a face-to-face, structured, clinical interview is light shed upon the manner (i.e., content and style) in which the Patient responds to and what, on the surface, ought to be clear and unambiguous, prima facie, emergency contact information.

Referral Information [Assessor to Rate Risk Factor Severity*]

It is necessary for the Assessor to accurately specify the referral source and referral contact information in order to substantiate the healthcare legitimacy of the NORA referral for the identified Patient and to be able to promptly provide directly to the referring entity a reliable and valid NORA protocol with summary impressions along with any concomitant recommendations regarding concurrent services.

Reason for Referral [Assessor to Rate Risk Factor Severity*]

The Assessor should inquire about the Patient's understanding of, and in their own words, the reason(s) for the current medical referral for an opioid risk assessment. For those Patients who do not clearly understand why a NORA referral has been made, it is important to determine the demonstrated ability of the Patient to comprehend the nature of the referral and the importance of assessing risk associated with opioid use.

If the construct of ‘risk’ is meaningless to the Patient, then it may be the case that they will not recognize potential ‘risk’ factors which could conceivably exacerbate or mitigate risk to themselves and/or to others. Endemic to any given response is the Patient's emerging and evolving acceptance of the need for their having “skin-in-the-game” in service of personal responsibility and personal accountability for working, in fidelitous collaboration, with their prescribing doctor(s) to decrease any and all untoward risk associated with opioid therapy.

Administrative Due Diligence Checklist [Assessor to rate Risk Factor Severity*) Each NORA-healthcare Assessor and/or their respective, healthcare agency needs to perform administrative due-diligence to ensure that accurate demographic, insurance, payment, referral and informed consents, if required, are carefully documented with all such information protected in accordance with applicable governmental, prevailing practice, and/or community standards governing the procurement, utilization, security, and retention of protected healthcare data. It is advisable to make explicit to the Patient, both in writing and verbally, thereafter, at the very beginning of the NORA assessment, of “NO GUARANTEE” of a favorable outcome from the opioid risk assessment. In so doing, the Assessor neither expresses nor implies that the administration of the NORA protocol is to be viewed merely a ‘rubber stamp’ of either the Patient's or prescriber's, a priori, motives for the assessment and any resultant outcome shall be a function of several sources of ‘subjective’ and ‘objective’ information, derived independently. Equally crucial, is the direct conveyance by the Assessor to the Patient of a relevant ‘expectancy set’ specific to the subsequent opioid risk assessment and planned Assessor evaluation structure going forward. This guidance is critical to communicate to the Patient the necessity of additional time to complete the NORA assessment, especially, if psychometric and/or medical testing is optionally administered or in the event that further inquiry of “critical items” or “noteworthy responses” identified by the Assessor is deemed pertinent to follow-up. Should psychometric and/or medical testing be administered to augment the face-to-face, structured, clinical NORA protocol, a Summary Report shall accompany the NORA, however, in virtually most every instance, the Assessor shall specify one of three possible NORA outcomes regarding new or continued opioid prescription therapy (RX), vis-a-vis, to: RECOMMEND, RECOMMEND WITH CAUTION or CANNOT RECOMMEND along with the possibility, if clinically warranted, of concurrent psychological and/or other designated healthcare services recommended as a condition of commencing, continuing, or withdrawing opioid RX. [N.B.: If concurrent services of any sort are recommended, per above, the Patient is under no obligation to receive said services from the NORA Assessor and/or their agency. The sole purpose of an Assessor identifying concurrent healthcare services by any authorized healthcare provider is to address and/or to mitigate potential or actual Patient ‘risk’ factors. Hence, such guidance is suggestive and not, necessarily, prescriptive]. ASSESSOR TO REQUEST OF PATIENT THE FOLLOWING INFORMATION

Medications [Assessor to Rate Risk Factor Severity*]

Next, the Patient is asked to identify all prescription (RX), as well as, non-prescription medications being currently used, as well as, their understanding for doctor's or self-prescription. At a minimum, this might include all prescription medications and any over-the-counter (OTC) medications, homeopathic/naturopathic remedies, Chinese or Ayurvedic or Native American Indian approaches, etc., along with any other topical, injected, inhaled, infused, or ingested ‘treatment’ (e.g., medical or non-medical cannabis, “designer drugs,” etc.).

Alcohol and Other Substances [Assessor to Rate Risk Factor Severity*]

In this next section of the NORA protocol, the Patient is requested to answer questions regarding their alcohol use. Also requested of the Patient is to identify or provide a list regarding any habitual Patient use of non-addictive or any potentially addictive substances. The purpose of these questions is to identify real or suspected use, abuse, tolerance, or dependence of either medically-prescribed and/or self-prescribed substances.

The involvement of a non-medical prescriber or unidentified influencer may be expressed or implied, for example, should one or more substances be recommended by an alternative or complementary practitioner of the healing arts or even by one's family member, friend, co-worker, ‘sponsor’ or internet influencer.

Medical Status [Assessor to Rate Risk Factor Severity*]

Next, the Assessor should inquire about any significant current and significant past medical status which, in the sole discretion of the Patient, may provide useful information concerning the Patient's medical concerns. However, non-lethal paper cuts, minor bumps and bruises, transient aches and pains or bouts of non-debilitating headaches or indigestion are not the primary focus of this section of the protocol, unless, of course, the Patient asserts that there is a pattern of chronicity, frequency, severity, amplitude, latency or duration of alleged symptoms, whether medical or psychological, which from the Patient's subjective experiential reality, represents a “significant” current or past medical concern to them.

Current and past medical concerns are to be queried next; including surgeries, hospitalizations, diseases, illnesses, injuries, etc., all of which are presumed to be significant. If the Patient tends to frequently identify relatively minor healthcare complaints, for which major healthcare services are sought, it is possible that the Patient may incorrectly report their symptoms or, perhaps, catastrophize them inaccurately; thus, calling into question their receptivity to opioids, as opposed to, consideration of a less-potent medicine.

The Patient is further queried to address their current use of opioids and/or narcotic pain medication. It will be ‘diagnostic’ as to how the Patient responds to these questions and the astute Assessor will provide the Patient ample opportunity to identify not only what specific medications are used but how such use affects the Patient's alleged complaints of pain.

Additional questioning by the Assessor focuses upon any habitual use by the Patient of non-addictive or potentially addictive substances. Responses may suggest a pattern and/or a tendency to “use” substances which may be harmful.

Psychological Status [Assessor to Rate Risk Factor Severity*)

Current significant mental health concerns and past significant mental health history information are to be elicited from the Patient including inquiry into signs and symptoms, either suggestive or indicative of, suspected probable imminent (or other) risk of harm to self and/or to others.

Clearly, an assessment of the Patient's current and previous mental health status relative to potential or actual risk factors is important, especially, since their use of opioids is presumed to have a direct impact upon brain-behavior functioning, inclusive of the potential for impaired functioning.

Inquiry into family-of-origin history of reported mental health issues may provide clinically heuristic inferences about “nature” and “nurture” causal or contributory effects upon the identified Patient. Also, it is necessary to assess whether or not the Patient is currently, and/or has been previously, in receipt of mental health or substance abuse treatment and, if so, where, when, and with whom.

Family [Assessor to Rate Risk Factor Severity*)

An inquiry of the Patient's family support system follows. “Family” may refer to the family one was born into, the family one grew up in, the family one creates along-the-way, etc. What is essential is for the Assessor to learn something meaningful about viable and enduring relational networks, whether actual family or “like family”, which serve to provide additional supervision, support and structure—if necessary—to the Patient for whom opioids are either being considered for prescription or are being medically titrated or altogether withdrawn.

Marital Status [Assessor to Rate Risk Factor Severity*)

Having the Patient address their current marital status, if applicable, and/or any previous marital status (inclusive of “common law” arrangements) is a logical extension of the assessment of their extant support system and, provisionally, allows the Assessor to make some reasonably educated inferences about the nature of the Patient's commitments, as well as, their perseverance to “stay-the-course” once they “give their word.”

Friends [Assessor to Rate Risk Factor Severity*]

The protocol section which follows is a logical extension for the Assessor to assess something useful of the nature regarding the Patient's social support system and whether or not there exists anyone else for the Patient to contact for assistance should the need arise. If a given Patient is bereft of family, friends or a significant other, then it would appear, a priori, that the attribution of prognosticated ‘risk’ might be, substantially, elevated.

Employment [Assessor to Rate Risk Factor Severity*]

Current and past employment history including any termination(s) and reason(s) is the next focus of the NORA protocol. The Assessor's inquiry allows for additional inferences about the Patient's suspected adherence or difficulty(ies) adhering to rule-governed behavior within the context of ‘normative’ standards of conduct. (Additional inquiry may be made by an astute Assessor regarding length of employment, promotions, demotions, suspensions, special recognition, awards or achievements, etc.)

Of course, there may be logical exceptions to these, a priori, clinical assumptions as may potentially occur with a Patient engaging in work-related (or societal) “civil disobedience,” unionizing, etc. However, the point of inquiry into employment history is to learn something meaningful about how the Patient ‘works’ with other people, superiors, subordinates, systems, policies, procedures, rules, regulations, requirements, etc.

Financial Status [Assessor to Rate Risk Factor Severity*]

Continuing in a similar vein, the Assessor interviews the Patient about current and past financial problems, including any filings or declarations of bankruptcy. Again, the purpose is not to penalize a person for experiencing serious financial difficulties, but rather to attempt to make an educated assessment about how the Patient addresses their financial responsibilities and accountabilities. In other words, does the Patient convey a recognition of the implications, with impact and consequence, of how their filing or declaring bankruptcy, might affect them, but also their creditors and the larger system-as-a-whole?

Legal Status [Assessor to Rate Risk Factor Severity*]

A similar concern continues with an inquiry into the Patient's current or past legal problems, including any pending litigation. If there exists a litigious pattern of either suing or being sued, the guidance to the opioid prescribing healthcare provider is to proceed with all due caution!

This may be, especially, the case if a given Patient proclaims, in an over-idealizing manner, how great a healthcare provider may be today, yet the avowed sentiment may quickly disintegrate into abject devaluation of the healthcare provider tomorrow, with the specter of a potential lawsuit to follow!

Arrest Status [Assessor to Rate Risk Factor Severity*]

Another line of questioning addresses any current and/or past history of arrests including charges, dates and legal disposition. In the event a Patient has a demonstrable criminal record, concerns about their suspected psychopathy, sociopathy, or anti-social behavior, notwithstanding, should the charges reveal drug-related offenses, then even greater caution is advised for the NORA Assessor, as well as, for the opioid-prescriber.

To reiterate, if the Patient affirmatively endorses any arrest experience, the prudent Assessor is strongly advised to specify the alleged transgressive behavior and any pertinent associated information (e.g., court adjudication, sentence, diversion, probation, neglect/truancy of restitution to victims, violation of parole, etc.) concerning the Patient's prior involvement.

Presumably, if the Patient has had prior involvement with the criminal justice system, there may be potential elevation of suspected opioid ‘risk’ associated with their abrogation of communal norms, inclusive of how they may, or may not, potentially, comply with medical advice.

Military Status [Assessor to Rate Risk Factor Severity*)

The section addressing the Patient's military background, if applicable, allows the Assessor to further gauge something useful about their inferred conformance to requirements, following orders and progressing or regressing in one's military career. Equally significant is the investigation of whether or not the Patient experienced active vs. inactive duty (i.e., was there deployment in a war-zone or not), as well as, whether or not their various civilian re-entry challenges remain an extant clinical issue for them; if their discharge was honorable or dishonorable and, finally, whether or not there was any, bona fide, service-connected disability and, if so, what the specific medical basis for the disability was/is.

Abuse/Neglect [Assessor to Rate Risk Factor Severity*)

What follows, thereafter, is an inquiry about the Patient being either an alleged victim and/or admitted perpetrator of any current and/or previous history of physical, emotional or sexual abuse and/or neglect. It is absolutely vital for the NORA Assessor to query the Patient about their being harmed, or even causing harm, since “pain” may be often defined in whatever way a person defines their pain and for those who are “in pain,” whatever means attenuates their pain may provide them sufficient justification to continue with whatever decreases their pain.

Strong Emotions [Assessor to Rate Risk Factor Severity*)

The Patient is next asked to address their typical modus operandi specific to their handling, or responding, to various stronger emotions, including, their experiencing loss, criticism and, of course, pain. To the extent the Patient conveys something positive and constructive about their own ‘self-agency’ concerning these areas of inquiry, then the Assessor may, generally, be less concerned about untoward ‘risk’ about the Patient.

However, to the extent that the Patient responds in any manner which reflects, either in their attitude or in their behavior a character or quality that they are, or may have significant potential to become, a raging, despairing, passive, dependent victim of fate and circumstance relative to their style of being-in-the-world, then the Assessor may have more serious cause for concern about ‘risk’.

Pain Status and Coping [Assessor to Rate Risk Factor Severity*]

Pain level ratings from zero-to-ten (0-10) are explored, where zero (0) represents the absence of any pain symptoms reported by the Patient and ten (10) represents intolerable pain. Related questions are designed to permit the Patient further elaboration of their complaints of symptomatology along with specification of pain localization, pain management and pain mitigation remedies.

It is necessary for the NORA Assessor to carefully explore reported pain levels with recourse to the Patient's subjective definition of their pain and, also, with recourse to the Patient's subjective experiential realities associated with their allegation of pain. This is essential because a Patient who claims to experience a pain level of “10” but consistently appears to function normally, with little-to-no manifest expression of pain or other limitations is, in all probability, not identical to a Patient who claims a pain level of “2” with demonstrably documented history of medical impairments (e.g., L2-S1 spinal fusion, median nerve damage, chronic headache secondary to post-traumatic sequelae to auto accident, etc.) and, despite few manifest expressions of pain or other limitations, simply carries-on and does what is necessary that needs to be done, no matter what their reported pain level. The Assessor should also review relevant cultural and/or multicultural considerations is assessing pain status.

Activities of Daily Living [Assessor to Rate Risk Factor Severity*]

Further corroboration of the Patient's reported pain level and what is done by the Patient to ameliorate their pain symptoms may be both expressed and implied by the Assessor inquiring about various activities of daily living (AOL's) and a brief sampling of various health-related problems which may be associated with or, otherwise, implicated by any drug use.

Another way of viewing this section's focus is for the Assessor to approximate an understanding of the Patient's adaptive behavioral functional competencies relative to their AOL's, as well as, to identify health-related problems which may be caused by, or which may be exacerbated by, the Patient's use of pharmacologies, inclusive of opioids. Additionally, to the extent there is a reported AOL-problem or health concern raised by the Patient, the Assessor may be in a unique position to recommend assistive and/or treatment services.

Firearms [Assessor to Rate Risk Factor Severity*]

The Assessor needs to carefully inquire about ownership and/or use of firearms and also if the Patient's legal right to own or use a firearm has ever been restricted and, if so, why. Common-sense must prevail insofar as opioid use may, or may not, impact the critical judgements a given Patient may make regarding when, where and under what legally appropriate circumstances one might responsibly use firearms. Where a Patient's ability to think, judge and act responsibly and accountably is, or previously has been, deleteriously impacted by opioid use, then all due caution is most likely required.

Optional High-Risk Inquiry [Assessor to Rate Risk Factor Severity*]

Several optional foci of potentially ‘high-risk’ clinical inquiry follow. It is imperative for the NORA Assessor to explicitly inform the Patient that the next four questions will be asked of them which they are absolutely under no obligation to answer; however, the questions will be asked, nonetheless. This caution, comparably-worded, should be reiterated for each of the optional questions to provide the Patient an opportunity to decline to respond in the event they feel uncomfortable with any line of specified inquiry. The experienced healthcare Assessor will readily appreciate the possible value of each ‘high-risk’ question as may shed meaningful light upon the Patient's religious concerns, their struggle with the specter of subjectively perceived, experienced and interpreted ‘sin’ along with any concomitant guilt, shame, humiliation, doubt, etc., and additional inquiries into the “worst thing” ever done by the Patient, as well as, to the Patient.

Narcotic Pain Medication Agreement [Assessor to Rate Risk Factor

Severity*)

The Assessor is then requested to directly ask the Patient, if applicable, if they are ready, willing and able to commit to a narcotic pain usage agreement (or contract) with their prescribing doctor on condition that one or more violations of the agreement may result in discontinuation of the prescribed medication(s). What is of clinical interest here is assessing the extent to which the Patient accepts personal responsibility and personal accountability for their role in the larger opioid ‘risk’ equation since, strictly speaking, all stakeholders share some of the responsibility and accountability.

Medical prescribers most, assuredly, seek to render a pain-relieving service, “good and true” whilst limiting untoward liability exposure and medical recipients of pain medication(s) most, assuredly, seek “good and true” healthcare services which may, hopefully, relieve the frequency, duration, chronicity, nature, amplitude, and latency of their pain.

Other Factors [Assessor to Rate Risk Factor Severity*)

If the Patient wishes to apprise the Assessor of any other factors pertinent to their NORA assessment, they are encouraged to do so. Space is also provided for any “miscellaneous” comments or notes, especially, since the NICA PSYCHOBIOSOCIAL RISK ASSESSMENT: DIAGNOSTIC INTERVIEW is designed to be an ‘organic’ clinical interview with plenty of latitude for the Assessor to ask a wide-array of questions deemed clinically relevant to an opioid risk assessment and for the Patient to answer in whatever individualized manner is relevant to them.

Attestation: Patient/Assessor The NICA PSYCHOBIOSOCIAL RISK ASSESSMENT: DIAGNOSTIC INTERVIEW portion of the NORA protocol is concluded upon obtaining the Patient's printed name, signature, and/or signature mark or authorization, and interview date followed by the Patient/Assessor documenting their names, credential(s), title or professional designation, license, registration or certification number, affiliation, and date interview was completed. If the Patient wishes to examine the document before signing, the guidance is for the Assessor to allow them to do so, however, any information recorded by the Assessor which the Patient does not concur with, shall oblige the Assessor to invite the Patient to provide a typed, printed or written emendation, albeit, prior to the end of the formal NORA protocol administration, if at all possible. Under no circumstances may a Patient alter what the Assessor has documented.

One embodiment example is a treatment protocol for assessing and managing pain based on a patient's risk of opioid misuse or addiction, comprising: a protocol tool; the protocol tool further comprises, a diagnostic interview, a DSM-5 or equivalent criteria, a DSM-5 related criteria, a summary report of the diagnostic interview, a summary report of DSM-5 or equivalent criteria, a summary report of DSM-5 related criteria, a summary diagnostic impression of the patient's risk based on the summary report of diagnostic interview, the summary report of DSM-5 or equivalent criteria, and the summary report of DSM-5 related criteria, a provisional consultative guidance for opioid treatment/therapy based on the summary diagnostic impression; and the provisional consultative guidance for opioid treatment/therapy based on the summary diagnostic impression further comprises an indication of recommend concurrent services, when applicable.

A second embodiment example is the treatment protocol of example one, wherein, the protocol tool further comprises: the diagnostic interview including an assessment of at least one psychobiosocial category, a valuation of risk factor severity for each assessment of the at least one psychobiosocial categories; the DSM-5 or equivalent criteria including a DSM-5 diagnostic criteria, a DSM-5 specifiers, a DSM-5 diagnosis and current severity; the summary report of the diagnostic interview including a total of all valuation of the risk factor severities assigned from the at least one psychobiosocial categories administered in the diagnostic interview, a risk factor severity quartile based on the total of all risk factor severities; and the summary report of DSM-5 or equivalent criteria, including an outcome of the DSM-5 diagnostic criteria, an outcome of the DSM-5 specifiers, and an outcome of the DSM-5 diagnosis and current severity.

A third embodiment example is the treatment protocol of example two, wherein the protocol tool further comprises an attestation which further includes a signature of the patient and the signature of the assessor.

A fourth embodiment example is the treatment protocol of example two, where in the protocol tool further comprises: the diagnostic interview with an assessment of at least one psychobiosocial category, further including a personal information category, an emergency contact category, a referral information category, a reason for referral category, an administrative due diligence checklist category, a medications category, an alcohol and other substances category, a medical status category, a psychological status category, a family category, a marital status category, a friends category, an employment category, a financial status category, a legal status category, an arrest status category, a military status category, abuse/neglect category, strong emotions category, pain status and coping category, activities of daily living category, firearms category, optional high-risk inquiry category, narcotic pain medication agreement category, and other factors category; the diagnostic interview with the valuation of risk factor severity for each assessment of the at least one psychobiosocial category/ies, further including a scale where Negligible=0, Low/Mild=1, Medium/Moderate=2, High/Severe=3, Extreme/Profound=4; the DSM-5 equivalent criteria with the DSM-5 diagnostic criteria further including a determination on whether the DSM-5 diagnostic criteria has been met; the DSM-5 related criteria further including an assessment of risk of harm: self criteria, an assessment of risk of harm: others criteria, an assessment of toxicology screen criteria, an assessment of additional medical considerations criteria, an assessment of additional queries regarding risk criteria, and a valuation of risk for each DSM-5 related criteria; and the summary report of DSM-5 related criteria further including the valuations of risk for each DSM-5 related criteria.

A fifth embodiment example is the treatment protocol of example four, where in the protocol tool further comprises: an attestation of the diagnostic interview further including a signature of the patient and the signature of the assessor.

By example, a method for using the treatment protocol tool of example one, for assessing and managing pain based on a patient's rick of opioid misuse or addition, comprises the steps of: an assessor to use the treatment protocol; a patient to provide information; the assessor performing the diagnostic interview; the assessor administering the DSM-5 or equivalent criteria; the assessor administering the DSM-5 related criteria; the assessor compiling the summary report of the diagnostic interview; the assessor compiling the summary report of DSM-5 or equivalent criteria; the assessor compiling the summary report of DSM-5 related criteria; the assessor indicating the summary diagnostic impression of the patient's risk based on the summary report of diagnostic interview, the summary report of DSM-5 or equivalent criteria, and the summary report of DSM-5 related criteria; the assessor indicating provisional consultative guidance for opioid treatment/therapy based on the summary diagnostic impression; and the assessor prescribing treatment and treating the patient accordingly.

By example, a method for using the treatment protocol tool of example five, for assessing and managing pain based on a patient's risk of opioid misuse or addiction, comprising: an assessor to use the treatment protocol; a patient to provide information; the assessor using the protocol tool of claim 5 in performing the diagnostic interview; the assessor obtaining attestation, by obtaining the signature of the patient and the signature of the assessor, while performing the diagnostic interview; the assessor administering the assessment of at least one psychobiosocial category, from the list of the following categories: personal information category, an emergency contact category, a referral information category, a reason for referral category, an administrative due diligence checklist category, a medications category, an alcohol and other substances category, a medical status category, a psychological status category, a family category, a marital status category, a friends category, an employment category, a financial status category, a legal status category, an arrest status category, a military status category, abuse/neglect category, strong emotions category, pain status and coping category, activities of daily living category, firearms category, optional high-risk inquiry category, narcotic pain medication agreement category, and other factors category; the assessor assigning a risk factor severity to each of the at least one psychobiosocial category/ies, using the scale where Negligible=0, Low/Mild=1, Medium/Moderate=2, High/Severe=3, Extreme/Profound=4; the assessor administering the DSM-5 equivalent criteria by applying: the DSM-5 diagnostic criteria to obtain a result, the DSM-5 specifiers to obtain a result, the DSM-5 diagnosis and current severity; the assessor administering the DSM-5 related criteria by assessing: the patient's risk of harm to themselves, the patient's risk of harm to others, the patient's toxicology screen, the patient's additional medical considerations, the patient's additional queries regarding risk criteria; the assessor compiling a summary report of the diagnostic interview by totaling all risk factor severities assigned from the at least one psychobiosocial categories administered and by assigning the risk factor severity quartile; the assessor compiling a summary report of the DSM-5 or equivalent criteria, indicating: the outcome of the DSM-5 diagnostic criteria, the outcome of the DSM-5 specifiers, the outcome of the DSM-5 diagnosis and current severity; the assessor compiling a summary report of DSM-5 related criteria by determining the valuations of risk for each of DSM-5 related criteria; the assessor indicating the summary diagnostic impression of the patient's risk based on the summary report of diagnostic interview, the summary report of DSM-5 or equivalent criteria, and the summary report of DSM-5 related criteria; the assessor indicating provisional consultative guidance for opioid treatment/therapy based on whether the assessor recommends, recommends with conditions, or cannot recommend opioid treatment/therapy; the assessor further indicating recommended concurrent services, if applicable; and the assessor prescribing treatment and treating the patient according to the provisional consultative guidance: if the guidance is to recommend opioid treatment, then prescribing the treatment to the patient, if the guidance is to recommend opioid treatment with conditions, then prescribing the treatment to the patient with the conditions indicated, and if the guidance is to not recommend opioid treatment, then prescribing non-opioid treatment to the patient.

What is claimed is:

1. A treatment protocol for assessing and managing pain based on a patient's risk of opioid misuse or addiction, comprising:
a protocol tool;
the protocol tool further comprises,
a diagnostic interview,
a DSM-5 or equivalent criteria,
a DSM-5 related criteria,
a summary report of the diagnostic interview,
a summary report of DSM-5 or equivalent criteria,
a summary report of DSM-5 related criteria,
a summary diagnostic impression of the patient's risk based on the summary report of diagnostic interview, the summary report of DSM-5 or equivalent criteria, and the summary report of DSM-5 related criteria,
a provisional consultative guidance for opioid treatment/therapy based on the summary diagnostic impression; and
the provisional consultative guidance for opioid treatment/therapy based on the summary diagnostic impression further comprises an indication of recommend concurrent services, when applicable.

2. The protocol tool of claim 1, further comprising:
the diagnostic interview including an assessment of at least one psychobiosocial category, a valuation of risk factor severity for each assessment of the at least one psychobiosocial categories;
the DSM-5 or equivalent criteria including a DSM-5 diagnostic criteria, a DSM-5 specifiers, a DSM-5 diagnosis and current severity;
the summary report of the diagnostic interview including a total of all valuation of the risk factor severities assigned from the at least one psychobiosocial categories administered in the diagnostic interview, a risk factor severity quartile based on the total of all risk factor severities; and
the summary report of DSM-5 or equivalent criteria, including an outcome of the DSM-5 diagnostic criteria, an outcome of the DSM-5 specifiers, and an outcome of the DSM-5 diagnosis and current severity.

3. The diagnostic interview of the protocol tool of claim 2, further comprises an attestation:
the attestation further comprises a signature of the patient and the signature of the assessor.

4. The protocol tool of claim 2, further comprising:
the diagnostic interview with an assessment of at least one psychobiosocial category, further including a personal information category, an emergency contact category, a referral information category, a reason for referral category, an administrative due diligence checklist category, a medications category, an alcohol and other substances category, a medical status category, a psychological status category, a family category, a marital status category, a friends category, an employment category, a financial status category, a legal status category, an arrest status category, a military status category, abuse/neglect category, strong emotions category, pain status and coping category, activities of daily living category, firearms category, optional high-risk inquiry category, narcotic pain medication agreement category, and other factors category;
the diagnostic interview with the valuation of risk factor severity for each assessment of the at least one psychobiosocial category/ies, further including a scale where Negligible=0, Low/Mild=1, Medium/Moderate=2, High/Severe=3, Extreme/Profound=4;
the DSM-5 equivalent criteria with the DSM-5 diagnostic criteria further including a determination on whether the DSM-5 diagnostic criteria has been met;
the DSM-5 related criteria further including an assessment of risk of harm: self criteria, an assessment of risk of harm: others criteria, an assessment of toxicology screen criteria, an assessment of additional medical considerations criteria, an assessment of additional queries regarding risk criteria, and a valuation of risk for each DSM-5 related criteria; and
the summary report of DSM-5 related criteria further including the valuations of risk for each DSM-5 related criteria.

5. The protocol tool of claim 4, further comprising
An attestation of the diagnostic interview further including a signature of the patient and the signature of the assessor;

6. A method for using the treatment protocol tool of claim 1, for assessing and managing pain based on a patient's risk of opioid misuse or addiction, comprising:
an assessor to use the treatment protocol;
a patient to provide information;
the assessor performing the diagnostic interview;
the assessor administering the DSM-5 or equivalent criteria;
the assessor administering the DSM-5 related criteria;
the assessor compiling the summary report of the diagnostic interview;
the assessor compiling the summary report of DSM-5 or equivalent criteria;
the assessor compiling the summary report of DSM-5 related criteria;
the assessor indicating the summary diagnostic impression of the patient's risk based on the summary report of diagnostic interview, the summary report of DSM-5 or equivalent criteria, and the summary report of DSM-5 related criteria;
the assessor indicating provisional consultative guidance for opioid treatment/therapy based on the summary diagnostic impression; and
the assessor prescribing treatment and treating the patient accordingly.

7. A method for using the treatment protocol tool of claim 5, for assessing and managing pain based on a patient's risk of opioid misuse or addiction, comprising:
an assessor to use the treatment protocol;
a patient to provide information;
the assessor using the protocol tool of claim 5 in performing the diagnostic interview;
the assessor obtaining attestation, by obtaining the signature of the patient and the signature of the assessor, while performing the diagnostic interview;
the assessor administering the assessment of at least one psychobiosocial category, from the list of the following categories: personal information category, an emergency contact category, a referral information category, a reason for referral category, an administrative due diligence checklist category, a medications category, an alcohol and other substances category, a medical status category, a psychological status category, a family category, a marital status category, a friends category, an employment category, a financial status category, a legal status category, an arrest status category, a military status category, abuse/neglect category, strong emotions category, pain status and coping category, activities of daily living category, firearms category, optional high-risk inquiry category, narcotic pain medication agreement category, and other factors category;
the assessor assigning a risk factor severity to each of the at least one psychobiosocial category/ies, using the scale where Negligible=0, Low/Mild=1, Medium/Moderate=2, High/Severe=3, Extreme/Profound=4;
the assessor administering the DSM-5 equivalent criteria by applying: the DSM-5 diagnostic criteria to obtain a result, the DSM-5 specifiers to obtain a result, the DSM-5 diagnosis and current severity;
the assessor administering the DSM-5 related criteria by assessing: the patient's risk of harm to themselves, the patient's risk of harm to others, the patient's toxicology screen, the patient's additional medical considerations, the patient's additional queries regarding risk criteria;
the assessor compiling a summary report of the diagnostic interview by totaling all risk factor severities assigned from the at least one psychobiosocial categories administered and by assigning the risk factor severity quartile;
the assessor compiling a summary report of the DSM-5 or equivalent criteria, indicating: the outcome of the DSM-5 diagnostic criteria, the outcome of the DSM-5 specifiers, the outcome of the DSM-5 diagnosis and current severity;
the assessor compiling a summary report of DSM-5 related criteria by determining the valuations of risk for each of DSM-5 related criteria;
the assessor indicating the summary diagnostic impression of the patient's risk based on the summary report of diagnostic interview, the summary report of DSM-5 or equivalent criteria, and the summary report of DSM-5 related criteria;
the assessor indicating provisional consultative guidance for opioid treatment/therapy based on whether the assessor recommends, recommends with conditions, or cannot recommend opioid treatment/therapy;
the assessor further indicating recommended concurrent services, if applicable; and
the assessor prescribing treatment and treating the patient according to the provisional consultative guidance: if the guidance is to recommend opioid treatment, then prescribing the treatment to the patient, if the guidance is to recommend opioid treatment with conditions, then prescribing the treatment to the patient with the conditions indicated, and if the guidance is to not recommend opioid treatment, then prescribing non-opioid treatment to the patient.
